(19) United States
(12) Patent Application Publication
(10) Pub. No.: US 2017/0174886 A1
(43) Pub. Date: Jun. 22, 2017

(54) Emulsions Of Branched Organopolysiloxanes

(71) Applicant: Dow Corning Corporation, Midland, MI (US)

(72) Inventors: SEVERINE CAUVIN, MONS (BE); LORRY DEKLIPPEL, PIETON (BE); YIHAN LIU, MIDLAND, MI (US); ROBERT D. KENNEDY, MIDLAND, MI (US)

(73) Assignee: Dow Corning Corporation, Midland, MI (US)

(21) Appl. No.: 15/116,091

(22) PCT Filed: Jan. 15, 2015
(86) PCT No.: PCT/US15/11543

§ 371 Date: Aug. 2, 2016

Related U.S. Application Data
(60) Provisional application No. 61/939,865, filed on Feb. 14, 2014.

(51) Int. Cl. C08L 83/06; A61K 8/892; C08G 77/38; A61Q 5/06; A61Q 19/00; A61K 8/06; A61Q 5/12

(52) U.S. Cl. CPC C08L 83/06; A61K 8/062; A61K 8/892; A61Q 5/12; A61Q 5/06; A61Q 19/00; C08G 77/38; C08L 2201/52; C08L 2203/02

(57) ABSTRACT

The invention relates to branched organopolysiloxanes and emulsions thereof, and to methods of preparation and uses of the branched organopolysiloxanes and emulsions thereof. A branched organopolysiloxane is prepared by the reaction of a branching agent with a substantially linear organopolysiloxane containing at least one hydroxyl or hydrolysable group bonded to silicon, in the presence of an inert fluid and a catalyst, such as a phosphazene catalyst. Phosphazene catalysts also have the advantage that the content of undesired low molecular weight cyclic silicones in the polymerisation product is low.

### CROSS REFERENCE TO RELATED APPLICATIONS

This application claims the benefit of U.S. Provisional Patent Application No. 61/939,865 as filed on Feb. 14, 2014, the content of which is incorporated herein by reference.

### FIELD OF THE INVENTION

This invention relates to branched organopolysiloxanes and emulsions thereof. The invention also relates to methods of making and uses of branched organopolysiloxanes and emulsions thereof.

### BACKGROUND OF THE INVENTION

Organopolysiloxanes have a wide variety of uses, for example, as sealants, antifoams, elastomers, pressure sensitive adhesives, or release agents. Organopolysiloxanes can be used in hair care and other personal care products, as well as in household care compositions.

Silicone emulsions can be made by processes, such as, (a) mechanical emulsification, or by (b) emulsion polymerization. Mechanical emulsification entails the homogenization of an oil phase, for example, a silicone polymer, and the aqueous phase to form a homogeneous emulsion. Emulsion droplet size (interchangeably used with particle size) depends on the type of surfactant used and the intensity of the homogenization. Mechanical emulsification typically requires considerable amount of energy input to break large droplets into smaller ones. In general, the higher the oil phase viscosity the more energy is required to break oil droplets. At higher oil phase viscosity, most conventional mixers fail to disperse the oil phase and break up the oil droplets into a size fine enough to result in stable emulsions. When the oil phase is of high viscosity, mixing of water and surfactant with the oil phase is difficult.

Emulsion polymerization, on the other hand, involves the simultaneous emulsification and polymerization of reactive monomers and/or oligomers in water. The advantage of emulsion polymerization is that monomers and oligomers usually have much lower viscosity, and therefore, the emulsification is less energy demanding. The drawback is that not every polymer can be synthesized by emulsion polymerization. There is only a limited range of emulsion polymerization chemistry applicable in practice. Furthermore, emulsion polymerization is limited to certain selections of surfactants and catalysts. The latter restriction is particularly severe, for example, when the catalyst deactivates in the presence of water.

The emulsification of silicones of high viscosity, such as, silicone gums, has for all practical purposes been limited to emulsion polymerization. In contrast, silicones with a low viscosity and hence a low molecular weight can easily be mechanically emulsified. Attempts to use mechanical methods for emulsifying silicone gums, such as, organopolysiloxane polymers of high molecular weight and viscosity, have largely been unsuccessful due to the above described problems.

Thus, there is a continuing need for improved hair care formulations. There is need for emulsions and methods of making emulsions of branched organopolysiloxanes that can be prepared by simple and inexpensive methods. The present invention provides methods for making aqueous emulsions of branched organopolysiloxane that have extremely high molecular weight and viscosity.

### SUMMARY OF THE INVENTION

The present invention provides methods for making oil-in-water emulsions comprising a branched organopolysiloxane, the method comprising:
(i) preparing a branched organopolysiloxane comprising reacting a branching agent with a substantially linear organopolysiloxane containing at least one hydroxyl or hydrolyzable group bonded to silicon in the presence of an inert fluid, a catalyst and optionally an end-blocking agent to obtain a solution or dispersion containing the branched organopolysiloxane, and a portion of the inert fluid;
(ii) quenching the reaction, if required;
(iii) adding water and one or more surfactants to the solution or dispersion containing the branched organopolysiloxane and mixing to form the oil-in-water emulsion; and
(iv) optionally applying shear to the emulsion to reduce particle size.

The present invention also provides emulsions comprising branched organopolysiloxanes, wherein the branched organopolysiloxanes are prepared by reacting a branching agent with a substantially linear organopolysiloxane containing at least one hydroxyl or hydrolyzable group bonded to silicon in the presence of an inert fluid, a catalyst, and optionally an end-blocking agent, wherein a portion of the inert fluid remains in the solution or dispersion containing the branched organopolysiloxane.

The emulsions of the invention can be used in personal care products, such as, those for application to the skin or hair.

### DETAILED DESCRIPTION OF THE INVENTION

The present invention provides simple and inexpensive methods to make aqueous emulsions of branched organopolysiloxane that have extremely high molecular weight and viscosity. The methods may use mechanical emulsification and accommodates a wide selection range of surfactants and catalysts. Energy consumption in emulsification is moderate considering the high viscosity of the polymer. This is achieved by incorporating an inert fluid before reaction occurs, and the polymerization and crosslinking reaction proceeds in the presence of the inert fluid. Emulsification is then made with the oil phase containing the high molecular weight branched organopolysiloxane and the inert fluid. The inert fluid according to the present invention is one which can function as a diluent and/or can form a solution or a homogeneous dispersion with the starting reactants, and/or does not create any undesired material property or performance of the final branched organopolysiloxane. The inert fluid may also be chosen to provide additional benefit to the performance of the final branched organopolysiloxane and/or the emulsions.

The use mechanical methods for emulsifying organopolysiloxane polymers of high molecular weight and viscosity, often referred to as silicone gums, presents problems. One way to overcome the problems is by diluting the highly viscous oil phase with a diluent fluid to form either a solution (in the case when the diluent is miscible with the oil) or dispersion (in the case when the diluent is immiscible with the oil). This solution to the problems may not always be useful because of the length of time it takes for a miscible diluent to solvate a high molecular weight polymer. In the case that a diluent fluid is immiscible with the high molecular weight polymer, a homogeneous mixture of the polymer and diluent may not occur since stirring a highly viscous polymer is difficult. Emulsions made of an inhomogeneous oil phase may not provide oil droplets that contain a representative and homogeneous composition.

The methods and emulsions of the present invention provide for the emulsification of high viscosity polymers. The methods and emulsions of the present invention also provide the advantages of using an inert fluid which can be substantially retained in the resulting solution or dispersion containing the branched organopolysiloxane. The inert fluid is believed to homogeneously distribute so that there is no phase separation. Each droplet in the final emulsion may contain a representative composition of the inert fluid. Furthermore, each droplet in the emulsion may contain a homogeneous blend of the branched organopolysiloxane and the inert fluid. This property of the emulsion droplets may be more desirable than in the case where an emulsion of the branched organopolysiloxane is blended with an emulsion of the inert fluid. For example, an article, such as, hair or skin, treated with the emulsion made according to the methods of the present invention is exposed to a homogeneous blend of the branched organopolysiloxane and the inert fluid. On the other hand, if the article is treated with an emulsion made by combining an emulsion of the branched organopolysiloxane with an emulsion of the inert fluid, the branched organopolysiloxane may be deposited in separate areas than the inert fluid resulting in less than optimum or undesirable properties and performance.

The term “portion” as used herein to describe that a portion of the inert fluid remains or is substantially retained in the solution or dispersion containing the branched organopolysiloxane means that all the inert fluid remains in the solution or dispersion, or 80% to 100% by weight, 90% to 100% by weight, 95% to 100% by weight, 98% to 100% by weight of the inert fluid remains in the solution or dispersion.

The term “substantial” or “substantially” as used herein to describe the substantially linear organopolysiloxane means that in relation to the notation of MDTQ of an organopolysiloxane, there is less than 5 mole % or less than 2 mole % of the units T and/or Q. The M, D, T, Q designate one (Mono), two (Di), three (Tri), or four (Quad) oxygen atoms covalently bonded to a silicon atom that is linked into the rest of the molecular structure. The M, D, T and Q units are typically represented as R^{e}ᵤSiO_{(4-u)/2}, where u is 3, 2, 1, and 0 for M, D, T, and Q, respectively.

The term “about” as used herein serves to reasonably encompass or describe minor variations in numerical values measured by instrumental analysis or as a result of sample handling. Such minor variations may be in the order of plus or minus 0% to 10% or plus or minus 0% to 5% of the numerical values.

The term “branched” as used herein describes a polymer with more than two end groups.

The term “comprising” is used herein in its broadest sense to mean and to encompass the notions of “include” and “consist of.”

The term “ambient temperature” or “room temperature” refers to a temperature between about 20° C. and about 30° C. Usually, room temperature ranges from about 20° C. to about 25° C.

The use of “for example” or “such as” to list illustrative examples does not limit to only the listed examples. Thus, “for example” or “such as” means “for example, but not limited to” or “such as, but not limited to” and encompasses other similar or equivalent examples.

The term “substituted” as used in relation to another group, for example, a hydrocarbon group, means, unless indicated otherwise, one or more hydrogen atoms in the hydrocarbon group has been replaced with another substituent. Examples of such substituents include, for example, halogen atoms such as chlorine, fluorine, bromine, and iodine; halogen atom containing groups such as chloromethyl, perfluorobutyl, trifluoroethyl, and nonafluorohexyl; oxygen atoms; oxygen atom containing groups such as (meth)acrylic and carboxyl; nitrogen atoms; nitrogen atom containing groups such as amines, amino-functional groups, amido-functional groups, and cyano-functional groups; sulphur atoms; and sulphur atom containing groups such as mercapto groups.

All viscosity measurements referred to herein were measured at 25° C. unless otherwise indicated.

An organopolysiloxane is intended to mean a polymer comprising multiple organosiloxane or polyorganosiloxane groups per molecule. Organopolysiloxane is intended to include polymers substantially containing only organosiloxane or polyorganosiloxane groups in the polymer chain, and polymers where the backbone contains both organosiloxane and/or polyorganosiloxane groups and organic polymer groups in the polymer chain. Such polymers may be homopolymers or copolymers, including, for example, block copolymers and random copolymers.

While the organopolysiloxane polymer has a substantially organopolysiloxane molecular chain, the organopolysiloxane polymer may alternatively contain a block copolymeric backbone comprising at least one block of siloxane groups and an organic component comprising any suitable organic based polymer backbone, for example, the organic polymer backbone may comprise, for example, polystyrene and/or substituted polystyrenes such as poly(α-methylstyrene), poly(vinylmethylstyrene), dienes, poly(p-trimethylsilylstyrene) and poly(p-trimethylsilyl-α-methylstyrene). Other organic components which may be incorporated in the polymeric backbone may include acetylene terminated oligophenylenes, vinylbenzyl terminated aromatic polysulphones oligomers, aromatic polyesters, aromatic polyester based monomers, polyalkylenes, polyurethanes, aliphatic polyesters, aliphatic polyamides and aromatic polyamides and the like.

The methods and emulsions of the present invention are useful in personal care products, particularly cosmetic formulations applied to skin or hair. Branched organopolysiloxanes have advantages over linear organopolysiloxanes. The branched organopolysiloxanes are useful due to their increased wash-off resistance compared to linear organosiloxanes as well as to their different, and often more pleasing, and superior sensory profile to touch, for example, when applied to hair and skin.

In the first step (i) of the methods of the present invention, a branched organopolysiloxanes may be prepared by reacting a branching agent with a substantially linear organopolysiloxane containing at least one hydroxyl or hydrolyzable group bonded to silicon in the presence of an inert fluid, a catalyst and optionally an end-blocking agent to obtain a solution or dispersion containing the branched organopolysiloxane, and a portion of the inert fluid. In one embodiment, the branched organopolysiloxanes may in general be prepared by a polycondensation reaction of a linear organopolysiloxane containing functional hydrolyzable groups, such as, Si—OH groups, with an alkoxysilane or other branching agents.

Another embodiment of the present invention provides methods for the preparation of a branched organopolysiloxane by the reaction of a branching agent with a substantially linear organopolysiloxane containing at least one hydroxyl or hydrolyzable group bonded to silicon in the presence of a phosphazene catalyst.

In the presence of a suitable branching agent having three or more reactive groups, the use of a phosphazene catalyst in the polycondensation reaction produces a branched organopolysiloxanes. Phosphazene catalysts also have other advantages, such as, that under certain conditions the content of undesired low molecular weight cyclic silicones in the final product is low.

In one embodiment of the present invention, the substantially linear organopolysiloxane (also referred herein as linear organopolysiloxane) generally contains on average more than one hydroxyl or hydrolyzable group bonded to silicon, such as, terminal hydroxyl or hydrolyzable groups. The substantially linear organopolysiloxane may have, for example, a general formula (1)

X¹-A-X² (1)

wherein X¹ and X² are independently selected from silicon containing groups which contain hydroxyl or hydrolyzable substituents and A represents a polymer chain. For example, X¹ or X² groups incorporating hydroxyl and/or hydrolyzable substituents include groups terminating with:
—Si(OH)₃; —(R^{a})Si(OH)₂; —(R^{a})₂SiOH; —(R^{a})Si(OR^{b})₂; —Si(OR^{b})₃; —(R^{a}₂)SiOR^{b}; or —(R^{a}₂)Si—R^{c}—SiR^{d}ₚ(OR^{b})₃₋ₚ
   wherein each R^{a} independently represents a monovalent hydrocarbyl group having from 1 to 8 carbon atoms, for example, an alkyl group such as methyl; R^{b} is an alkyl; and R^{d} is an alkyl or alkoxy group, wherein the alkyl and alkoxy groups have 1 to 6 carbon atoms; R^{d} is a divalent hydrocarbon group having 1 to 8 carbon atoms which may be interrupted by one or more siloxane spacers having 1 to 6 silicon atoms; and p has the value 0, 1 or 2. Groups X¹ and X² can also be terminal groups of the formula —(R^{a})₂SiOH. The linear organopolysiloxane may include a small amount, for example, less than 20%, of a non-reactive terminal groups of the formula R^{a}₃SiO_{1/2}.

In one embodiment, the polymer chain A can be a polydiorganosiloxane chain comprising siloxane units of formula (2)

—(R²₂SiO)— (2)

wherein each R² is independently an organic group such as a hydrocarbon group having from 1 to 18 carbon atoms, a substituted hydrocarbon group having from 1 to 18 carbon atoms or a hydrocarbonoxy group having 1 to 18 carbon atoms.

Examples of hydrocarbon groups R² include, for example, methyl, ethyl, propyl, butyl, vinyl, cyclohexyl, phenyl and tolyl groups. Substituted hydrocarbon groups have one or more hydrogen atoms in a hydrocarbon group replaced with another substituent, for example, a halogen atom such as chlorine, fluorine, bromine or iodine, an oxygen atom containing group such as acrylic, methacrylic, alkoxy or carboxyl, a nitrogen atom containing group such as an amino, amido or cyano group, or a sulphur atom containing group such as a mercapto group. Examples of substituted hydrocarbon groups include a propyl group substituted with chlorine or fluorine such as 3,3,3-trifluoropropyl, chlorophenyl, beta-(perfluorobutyl)ethyl or chlorocyclohexyl group. In some embodiments, at least some or all of the R² groups are methyl. The polydiorganosiloxanes can be polydialkylsiloxanes, for example, polydimethylsiloxanes.

The polydiorganosiloxane chain comprising units of the formula (2) may be homopolymers or copolymers. Mixtures of different polydiorganosiloxanes are also suitable. In the case of polydiorganosiloxane copolymers, the polymer chain may comprise a combination of blocks made from chains of units depicted in formula (2) above where the two R² groups are:
both alkyl groups (such as, methyl or ethyl), or
alkyl and phenyl groups, or
alkyl and fluoropropyl, or
alkyl and vinyl or
alkyl and hydrogen groups.

Typically, at least one block will comprise siloxane units in which both R² groups are alkyl groups.

The polymer A may alternatively have a block copolymeric backbone comprising at least one block of siloxane groups of the type depicted in formula (2) above and at least one block comprising any suitable organic polymer chain. Examples of suitable organic polymer chains can be polyacrylic, polyisobutylene and polyether chains.

The substantially linear organopolysiloxane generally has a degree of polymerization such that its viscosity at 25° C. is between 5 mPa·s and 5000 mPa·s, or between 10 mPa·s and 500 mPa·s.

The branching agent is a compound that contains three or more reactive groups. The branching agent may be a reactive silane having more than two reactive groups capable of hydrolyzing and condensing with itself and with the linear organopolysiloxane containing at least one hydroxyl or hydrolysable group bonded to silicon. The branching agent which reacts with the linear organopolysiloxane contains an average of more than two silicon-bonded hydrolyzable groups per molecule.

In one embodiment of the present invention, the branching agent may have a general formula

R¹Si(OR)₃

wherein R is selected from the group consisting of hydrogen, an alkyl group of 1 to 6 carbon atoms, an alkenyl group of 2 to 6 carbon atoms, a saturated or unsaturated cyclic group of 3 to 6 carbon atoms, an acyl group of 1 to 6 carbon atoms, and an aryl-carbonyl group wherein the aryl is of 6 to 10 carbon atoms, wherein the alkyl, alkenyl, cyclic or aryl group is unsubstituted or substituted with one or more groups selected from an alkyl group of 1 to 6 carbon atoms, a hydroxy, an alkoxy group of 1 to 6 carbon atoms, a cycloalkyl group of 3 to 6 carbon atoms, halogen and cyano, and R¹ is a monovalent substituted or unsubstituted hydrocarbon group of 1 to 18 carbon atoms or an alkoxy group of 1 to 6 carbon atoms. The R group may be, for example, CH₃C(O)—, CH₃CH₂C(O)—, HOCH₂CH₂—, CH₃OCH₂CH₂—, or C₂H₅OCH₂CH₂.

In one embodiment of the present invention, the branching agent is an alkylalkoxysilane. The alkoxy group can have 1 to 4 carbon atoms. For example, alkoxy group can be methoxy or ethoxy group. In one embodiment of the present invention, R¹ includes alkyl groups, for example, methyl, ethyl, propyl, butyl, hexyl, octyl, 2-ethylhexyl, lauryl or stearyl; cycloalkyl groups, for example, cyclopentyl or cyclohexyl; alkenyl groups, for example vinyl, allyl or hexenyl; aryl groups, for example, phenyl or tolyl; aralkyl groups, for example, 2-phenylethyl; and groups obtained by replacing all or part of the hydrogen in the preceding organic groups with halogen, for example, 3-chloropropyl, 3,3,3-trifluoropropyl.

In one embodiment of the present invention, the branching agent is trialkoxysilanes, such as, methyltrimethoxysilane, methyltriethoxysilane, isobutyltrimethoxysilane, n-octyltriethoxysilane, n-octyltrimethoxysilane, ethyltrimethoxysilane, ethyltriethoxysilane, vinyltrimethoxysilane, vinyltriethoxysilane, phenyltrimethoxysilane and 3,3,3-trifluoropropyltrimethoxysilane. Trialkoxysilanes having a long chain alkyl group R¹, for example, 6 to 18 carbon atoms, react with the linear organopolysiloxane to form a branched organopolysiloxane having a long chain alkyl group at the branching point. For example, if n-octyltrimethoxysilane is the used as the branching agent, an octyl group would be at the branching position. The presence of such a long chain alkyl group increases the compatibility of the branched organopolysiloxane with organic materials, for example, hydrocarbon solvents or organic polymers.

The branching agent may alternatively be a tetraalkoxysilane such as, tetraethoxysilane (tetraethyl orthosilicate). Reaction of the linear organoplysiloxane with a tetraalkoxysilane may form a branched organopolysiloxane having Si-alkoxy functionality in the organopolysiloxane chain as well as in the branching point. The branching agent may alternatively be a mixture of trialkoxysilane and tetraalkoxysilane.

In another embodiment, the branching agent can be a hydrolysis derivative of a silane or a partially condensed version in which some reactive groups have been hydrolyzed and condensed to form siloxane linkages, and the other reactive groups still remain bonded to the silicon. Such a partially condensed silane contains on average more than two reactive groups per molecule bonded to silicon. Such a hydrolysis derivative of a silane may be, for example, an oligomer partially condensed trialkoxysilane. Such an oligomer may have a branched structure as well as Si-alkoxy groups to provide further branching sites. Tetraalkoxysilanes may also be used in partially condensed form; for example, partially condensed tetraethoxysilane containing SiO₂ branching units.

The branching agent and the substantially linear organopolysiloxane containing at least one hydroxyl or hydrolyzable group bonded to silicon may be reacted in amounts such that the molar ratio of Si-bonded reactive groups in the branching agent to hydroxyl or hydrolyzable groups in the substantially linear organopolysiloxane is from 1:100 to 1:1, or 1:40 to 1:2. If the substantially linear organopolysiloxane has hydrolyzable groups other than hydroxyl groups, it may be suitable for a controlled amount of moisture to be present during the reaction. The branched organopolysiloxane may contain reactive terminal Si—OH or Si-alkoxy groups.

The catalyst is any catalyst that would catalyze condensation reaction between silanols in the linear organopolysiloxane, and between the branching agent and the substantially linear organopolysiloxane containing at least one hydroxyl or hydrolysable group bonded to silicon.

In one embodiment of the present invention, the catalyst may be a phosphazene catalyst that generally contains at least one —(N═P<)— unit and is usually an oligomer having up to 10 such phosphazene units, for example, having an average of from 1.5 up to 5 phosphazene units. The phosphazene catalyst may be, for example, a halophosphazene, particularly a chlorophosphazene (phosphonitrile chloride), an oxygen-containing halophosphazene, a phosphazene base or an ionic derivative of a phosphazene such as a phosphazenium salt, particularly an ionic derivative of a phosphonitrile halide such as a perchlorooligophosphazenium salt.

In one embodiment, the phosphazene catalyst is an oxygen-containing halophosphazene, particularly an oxygen-containing chlorophosphazene. Such an oxygen-containing chlorophosphazene may have, for example, the formula Cl(PCl₂═N)ₙ—P(O)Cl₂ or HO(PCl₂═N)ₙ—P(O)Cl₂. The average value of n may be, for example, an integer in the range 1 to 10, particularly 1 to 5. The catalyst may also comprise tautomers of the catalyst of the formula HO(PCl₂═N)ₙ—P(O)Cl₂. For example, a tautomer of the catalyst may be P(O)Cl₂NH(PCl₂═N)ₙ—P(O)Cl₂, wherein n is an integer in the range 0 to 10. Another type of suitable oxygen-containing chlorophosphazene has the formula Z¹O(PCl₂═N)ₙ—P(O)Cl₂ in which Z¹ represents an organosilicon radical bonded to phosphorus via oxygen, for example, a phosphazene catalyst of the formula R⁵₃SiO(PCl₂═N)ₙ—P(O)Cl₂ where each R⁵ represents a monovalent hydrocarbon or substituted hydrocarbon group having 1 to 18 carbon atoms. The catalyst may also comprise condensation products of such an organosilicon-containing phosphazene. All or some of the chlorine atoms in any of the above oxygen-containing phosphazenes may be replaced by radicals Q, in which Q represents the hydroxyl group, monovalent organic radicals, such as alkoxy radicals or aryloxy radicals, halogen atoms other than chlorine, organosilicon radicals and phosphorus-containing radicals.

In another embodiment, the phosphazene catalyst is a perchlorooligophosphazenium salt of formula

[Cl₃P—(N═PCl₂)ₙCl]⁺Z

where n has an average value in the range 1 to 10 and Z represents an anion. The anion may be a complex anion and may be, for example, of the formula MXᵥ₊₁ in which M is an element having an electronegativity on Pauling's scale of from 1.0 to 2.0 and valency v and X is a halogen atom. The element M may be, for example, phosphorus or antimony. The anion Z may alternatively be a complex anion of the formula [MX_{v-y+1}R³_{y}]— wherein R³ is an alkyl group having 1 to 12 carbon atoms and y has a value between 0 and v, as described in U.S. Pat. No. 5,457,220, which is incorporated by reference in its entirety.

In one embodiment, the phosphazene catalyst may be a hydrolyzed phosphazene catalyst or a non-hydrolyzed phosphazene catalyst. The phosphazene catalyst may alternatively be a phosphazene base, such as, an aminated phosphazene as described in U.S. Pat. No. 6,001,928, U.S. Pat. No. 6,054,548 or U.S. Pat. No. 6,448,196, all of which are incorporated by reference in their entirety. Such a phosphazene base may be formed by reaction of a perchlorooligophosphazenium salt with a secondary amine followed by ion exchange reaction with a basic nucleophile. The secondary amine is, for example, of formula HNR⁴₂, and some or all of the chlorophosphazene oligomer are replaced by —NR⁴₂ groups.

The catalyst may typically be present at 1 to 200 parts per million based on the combined weight of the branching agent and substantially linear organopolysiloxane. For example, a phosphazene catalyst may typically be present at 1 to 200 parts per million or at 5 to 50 parts per million based on the combined weight of the branching agent and substantially linear organopolysiloxane. The reaction between the branching agent and substantially linear organopolysiloxane may be carried out at ambient temperature but may also be carried out at an elevated temperature, for example, in the range 50° C. to 100° C.

The extent of polymerization in the methods of the present invention is such that the branched organopolysiloxane produced has a weight average molecular weight (Mw) at least five times, at least ten times, at least fifty times, at least one hundred times, at least two hundred times, at least three hundred times, at least four hundred times, or at least five hundred times the Mw of the starting linear organopolysiloxane. For example, the branched organopolysiloxane may have a Mw between five times to five thousand times the Mw of the starting linear organopolysiloxane. The Mw may be measured by gel permeation chromatography (GPC). The Mw of the branched organopolysiloxane produced may be at least 10,000 g/mol, at least 100,000 g/mol, and may be as high as 1,000,000 g/mol or more.

In some embodiments of the present invention, the branched organopolysiloxane may exhibit high polydisperisty. In another embodiment, the number average molecular weight (Mn) of the branched organopolysiloxane may be from about 1,000 to about 1,000,000 g/mol; or about 100,000 g/mol. In another embodiment, the Mw of the branched organopolysiloxane is from about 10,000 to about 10,000,000 g/mol, or about 1,000,000 g/mol. In another embodiment, the Z-number average molecular weight (Mz) from about 40,000 to about 40,000,000 g/mol, or about 4,000,000 g/mol.

In another embodiment, the Mw of the linear organopolysiloxane starting material may be from about 1,000 to about 6,000 g/mol, or about 3,500 g/mol.

The reaction between the branching agent and substantially linear organopolysiloxane is carried out in the presence of an inert fluid. The inert fluid is a non-reactive fluid, that is, it does not participate in the reaction between the branching agent and the substantially linear organopolysiloxane. The inert fluid itself may bring additional benefits to the final branched organopolysiloxane or the emulsions. The inert fluid may be, for example, an organic based inert fluid and is generally chosen to have no groups reactive with the branching agent or with the substantially linear organopolysiloxane.

In one embodiment of the present invention, the inert fluid is a liquid. A liquid inert fluid provides advantages that include, among others, the formation of very high molecular weight branched polymers and the formation of flowable products for easy handling. A liquid inert fluid may be, for example, a solvent for the substantially linear organopolysiloxane and/or the branching agent, or may be a non-solvent. The inert fluid may be, for example, an liquid organic based inert fluid and is generally chosen to have no groups reactive with the branching agent or with the substantially linear organopolysiloxane.

Any suitable inert fluid or combination of inert fluids may be used in the methods and emulsions of the present invention. Suitable inert fluids are ones that either dissolve the substantially linear organopolysiloxane forming a clear solution or can be mixed with the linear organopolysiloxane to form a homogeneous dispersion without phase separation within a timeframe of the reaction and subsequent emulsification. Any of the fluids described as extenders in WO2006/106362, which is incorporated by reference in its entirety, may be used as an inert fluid. The inert fluid may be, for example, any one or combination of the following:
hydrocarbon oils such as mineral oil fractions comprising linear (e.g., n-paraffinic) mineral oils, branched (iso-paraffinic) mineral oils, and/or cyclic (sometimes referred to as naphthenic) mineral oils, the hydrocarbons in the oil fractions comprising from 5 to 25 carbon atoms per molecule, or a liquid linear or branched paraffin containing 12 to 40 carbon atoms;
trialkylsilyl terminated polydialkyl siloxane where the alkyl groups may be the same or different and comprises from 1 to 6 carbon atoms, for example, a methyl group, and have a viscosity of from 100 to 100000 mPa·s at 25° C. or from 1000 to 60000 mPa·s at 25° C.;
polyisobutylenes (PIB);
phosphate esters such as trioctyl phosphate;
polyalkylbenzenes, linear and/or branched alkylbenzenes such as heavy alkylates, dodecyl benzene and other alkylarenes;
esters of aliphatic monocarboxylic acids;
linear or branched mono unsaturated hydrocarbons such as linear or branched alkenes or mixtures thereof containing from 8 to 25 carbon atoms;
natural oils and derivatives thereof; and
the fluids described as extenders in WO2006/106362, which is incorporated by reference in its entirety.

In one embodiment, the inert fluids include the mineral oil fractions, natural oils and alkylcycloaliphatic compounds and alkybenzenes including polyalkylbenzenes. Any suitable mixture of mineral oil fractions may be used as the inert fluid. For example, inert fluids include alkylcyclohexanes of molecular weight above 220, paraffinic hydrocarbons and mixtures thereof containing from 1% to 99%, or from 15% to 80% n-paraffinic and/or isoparaffinic hydrocarbons (linear branched paraffinic) and 1% to 99%, or 20% to 85% cyclic hydrocarbons (naphthenic) and a maximum of 3%, or a maximum of 1% aromatic carbon atoms. The cyclic paraffinic hydrocarbons may be monocyclic and/or polycyclic hydrocarbons (naphthenics).

In another embodiment, the inert fluid may be a natural oil. Natural oils are oils derived from animals, seeds or nuts and not from petroleum. Such natural oils are generally triglycerides of mixtures of fatty acids, particularly mixtures containing some unsaturated fatty acid. Inert fluids containing natural oils may be, for example, preferred for use in some personal care products. The inert fluid may be a derivative of a natural oil such as a transesterified vegetable oil, a boiled natural oil, a blown natural oil, or a stand oil (thermally polymerized oil).

The alkylbenzene compounds suitable for use as inert fluids include, for example, heavy alkylate alkylbenzenes and alkylcycloaliphatic compounds. Inert fluids include, for example, alkyl substituted aryl compounds which have aryl groups, such as benzene substituted by alkyl and/or other substituents, and a molecular weight of at least 200. Examples of inert fluids can be the extenders described in U.S. Pat. No. 4,312,801, which is incorporated by reference in its entirety.

In one embodiment of the present invention, the amount of the inert fluid may be from 1% to 80%, or 25% to 60% of the combined weight of the branching agent, the substantially linear organopolysiloxane and the inert fluid. Other non-reactive additives whose presence provides additional benefit in specific applications, for example, heat stabilizers, flame retardants, UV stabilizers, fungicides, biocides or perfumes, may be dissolved in the inert fluid.

In another embodiment of the present invention, the ratio of the linear organopolysiloxane to the inert fluid may be from about 1:10 to about 10:1 by weight. For example, the ratio can be 1:1, 3:2, 7:3, 4:1, 1:9, 2:3, 3:7, 1:4 or 9:1.

The inert fluid may be a silicone compound having organic groups such that the inert fluid is not reactive with the branching agent or with the substantially linear organopolysiloxane. For example, the inert fluid may be a trialkylsilyl terminated polydialkyl siloxane, wherein each alkyl group may be the same or different and comprises from 1 to 6 carbon atoms. Alternatively, the alkyl groups are methyl groups. The viscosity is from 100 to 100000 mPa·s at 25° C. or from 1000 to 60000 mPa·s at 25° C.

The inert fluid may alternatively be a solid such as a wax, having a melting point in the range 30° C. to 100° C. The wax may be, for example, a hydrocarbon wax such as a petroleum-derived wax, or a wax comprising carboxylic esters such as beeswax, lanolin, tallow, carnauba, candelilla, tribehenin or a wax derived from plant seeds, fruits, nuts or kernel, including softer waxes referred to as ‘butter,’ for example, mango butter, shea butter or cocoa butter. The wax may alternatively be a polyether wax or a silicone wax.

The optional end-blocking agents may be, for example, low molecular weight trialkylsilyl-terminated polydialkyl siloxane, hexamethyldisilazane, an trialkylmonoalkoxysilane (R¹₃SiOR), trialkymonoacyloxysilane (R¹₃SiO₂CR), wherein R and R¹ are as defined above, or linear or branched alcohols, such as, methanol, ethanol, propanol, ISOFOL® alcohols. The amount of the optional end-blocking agent can be used in stoichiometric amounts so as to produce a branched organopolysiloxane having a Mw between five times to five thousand times the Mw of the starting linear organopolysiloxane, and will be apparent to one skilled in the art depending on the exact final molecular weight of the branched organopolysiloxane to be prepared. For example, the optional end-blocking agent and the substantially linear organopolysiloxane containing at least one hydroxyl or hydrolyzable group bonded to silicon may be reacted in amounts such that the molar ratio of the end-blocking groups in the end-blocking agent to the hydroxyl or hydrolyzable groups in the substantially linear organopolysiloxane is from 1:10,000 to 1:1, or 1:1,000 to 1:2, or 1:200 to 1:10.

In one embodiment of the present invention, the branching agent is methyltrimethoxysilane, methyltriacetoxysilane, ethyltriacetoxysilane, or tetraethyl orthosilicate, or combinations thereof. Other branching agents that can be used in some embodiments of the present invention may be, for example, a silane or a hydrolyate or condensation product of. The branching agent should contain three or more reactive sites on the molecule. The branching agent may alternatively be an organic polymer substituted by silicon-bonded hydrolysable groups having three or more reactive sites per molecule.

The hydrolysable groups in the branching agent may be, for example, selected from acyloxy groups (for example, acetoxy, octanoyloxy, and benzoyloxy groups); ketoximino groups (for example dimethyl ketoximo, and isobutylketoximino); alkoxy groups (for example methoxy, ethoxy, an propoxy) and/or alkenyloxy groups (for example isopropenyloxy and 1-ethyl-2-methylvinyloxy).

When the branching agent is a silane having three silicon-bonded hydrolysable groups per molecule, the fourth group is suitably a non-hydrolysable silicon-bonded organic group. These silicon-bonded organic groups are suitably hydrocarbyl groups which are optionally substituted by halogen such as fluorine and chlorine. Examples of such fourth groups include alkyl groups (for example methyl, ethyl, propyl, and butyl); cycloalkyl groups (for example cyclopentyl and cyclohexyl); alkenyl groups (for example vinyl and allyl); aryl groups (for example phenyl, and tolyl); aralkyl groups (for example 2-phenylethyl) and groups obtained by replacing all or part of the hydrogen in the preceding organic groups with halogen. The fourth silicon-bonded organic group mat be methyl or ethyl.

Examples of branching agents include acyloxysilanes, particularly acetoxysilanes such as methyltriacetoxysilane, vinyltriacetoxysilane, ethyl triacetoxysilane, di-butoxy diacetoxysilane and/or dimethyltetraacetoxydisiloxane, and also phenyl-tripropionoxysilane. The branching agent may be an oxime-functional silane such as methyltris(methylethylketoximo)silane, vinyl-tris(methylethylketoximo)silane, or an alkoxytrioximosilane. The branching agent may be an alkoxysilane, for example, an alkyltrialkoxysilane such as methyltrimethoxysilane, methyltriethoxysilane, isobutyltrimethoxysilane or ethyltrimethoxysilane, an alkenyltrialkoxysilane such as vinyltrimethoxysilane or vinyltriethoxysilane, or phenyltrimethoxysilane, 3,3,3-trifluoropropyltrimethoxysilane, or ethylpolysilicate, n-propylorthosilicate, ethylorthosilicate, or an alkenyloxysilane, such as, methyltris(isopropenoxy)silane or vinyltris(isopropenoxy)silane. The branching agent may alternatively be a short chain polydiorganosiloxane, for example, polydimethylsiloxane, tipped with trimethoxysilyl groups or may be an organic polymer, for example, a polyether such as polypropylene oxide, tipped with methoxysilane functionality such as trimethoxysilyl groups. The branching agent used may also comprise any combination of two or more of the above.

Further alternative branching agents include alkylalkenylbis(N-alkylacetamido) silanes such as methylvinyldi-(N-methylacetamido)silane, and methylvinyldi-(N-ethylacetamido)silane; dialkylbis(N-arylacetamido) silanes such as dimethyldi-(N-methylacetamido)silane; and dimethyldi-(N-ethylacetamido)silane; alkylalkenylbis(N-arylacetamido) silanes such as methylvinyldi(N-phenylacetamido)silane and dialkylbis(N-arylacetamido) silanes such as dimethyldi-(N-phenylacetamido)silane, or any combination of two or more of the above.

The amount of branching agent present will depend upon the particular nature of the branching agent, particularly its molecular weight. The methods of the present invention uses branching agent in at least a stoichiometric amount as compared to the linear organopolysiloxane. The compositions may contain, for example, from 0.05% to 10% by weight of the branching agent, generally from 0.1% to 10% by weight per weight of the linear organopolysiloxane. For example, acetoxysilane or oximinosilane branching agent may typically be present in amounts of from 3% to 8% by weight.

In one embodiment of the present invention, the ratio of the linear organopolysiloxane to the branching agent may be from about 10:1 to about 1000:1, or 100:1 to about 500:1 by weight. In another embodiment, the ratio may be from about 200:1 to about 400:1 by weight, or about 300:1 by weight. In another embodiment, the molar ratio of the linear organopolysiloxane to the branching agent at the beginning of the reaction may be from about 5:1 to about 20:1 at a DP (degree of polymerization of 40), from about 10:1 to about 15:1 at a DP of 40, or about 13:1 at DP of 40.

In step (ii) of the methods of the present invention, the reaction between the branching agent and the linear organopolysiloxane may be quenched, if required, after a desired degree of polymerization has been achieved. Quenching means termination of the reaction by, for example, adding a neutraliser when a desired degree of polymerization has been reached. The neutralizer may, for example, be a trialkylamine as described in U.S. Pat. No. 5,457,220, which is incorporated by reference in its entirety.

The oil phase produced after quenching, or as a result of the reaction in step (i), comprising a branched organopolysiloxane and an inert diluent fluid.

In step (iii) of the methods of the present invention, any suitable surfactant or combination of surfactants may be utilised. The surfactant may in general be a non-ionic surfactant, a cationic surfactant, an anionic surfactant, or an amphoteric surfactant, although not all procedures for carrying out the methods of the present invention may be used with all surfactants. The amount of surfactant used will vary depending on the surfactant, but generally is up to about 30% by weight based on the weight of the oil phase containing the branched organopolysiloxane and the inert fluid.

Examples of non-ionic surfactants include condensates of ethylene oxide with long chain fatty alcohols or fatty acids such as a alcohol having 12 to 16 carbon atoms, condensates of ethylene oxide with an amine or an amide, condensation products of ethylene and propylene oxide, esters of glycerol, sucrose, sorbitol, fatty acid alkylol amides, sucrose esters, fluoro-surfactants, fatty amine oxides, polyoxyalkylene alkyl ethers such as polyethylene glycol long chain (12 to 14 carbon atoms) alkyl ether, polyoxyalkylene sorbitan ethers, polyoxyalkylene alkoxylate esters, polyoxyalkylene alkylphenol ethers, ethylene glycol propylene glycol copolymers and alkylpolysaccharides, for example, materials of structure R²⁴—O—(R²⁵O)ₛ-(G)ₜ wherein R²⁴ represents a linear or branched alkyl group, a linear or branched alkenyl group or an alkylphenyl group, R²⁵ represent an alkylene group, G represents a reduced sugar, s denotes 0 or a positive integer and t represent a positive integer as described in U.S. Pat. No. 5,035,832, which is incorporated by reference in its entirety. Non-ionic surfactants additionally include polymeric surfactants such as polyvinyl alcohol (PVA) and polyvinylmethylether.

Representative examples of suitable commercially available non-ionic surfactants include polyoxyethylene fatty alcohols sold under the tradename BRIJ® by Croda. Some examples are BRIJ® L23, an ethoxylated alcohol known as polyoxyethylene (23) lauryl ether, and BRIJ® L4, another ethoxylated alcohol known as polyoxyethylene (4) lauryl ether. Some additional non-ionic surfactants include ethoxylated alcohols sold under the trademark TERGITOL® by The Dow Chemical Company, Midland, Mich. Some example are TERGITOL® TMN-6, an ethoxylated alcohol known as ethoxylated trimethylnonanol; and various of the ethoxylated alcohols, i.e., the 12-14 carbon atoms secondary alcohol ethoxylates, sold under the trademarks TERGITOL® 15-S-5, TERGITOL® 15-S-12, TERGITOL® 15-S-15, and TERGITOL® 15-S-40. Surfactants containing silicon atoms may also be used.

Examples of suitable amphoteric surfactants include imidazoline compounds, alkylaminoacid salts, and betaines. Specific examples include cocamidopropyl betaine, cocamidopropyl hydroxysulfate, cocobetaine, sodium cocoamidoacetate, cocodimethyl betaine, N-coco-3-aminobutyric acid and imidazolinium carboxyl compounds. Representative examples of suitable amphoteric surfactants include imidazoline compounds, alkylaminoacid salts, and betaines.

Examples of cationic surfactants include quaternary ammonium hydroxides such as octyl trimethyl ammonium hydroxide, dodecyl trimethyl ammonium hydroxide, hexadecyl trimethyl ammonium hydroxide, octyl dimethyl benzyl ammonium hydroxide, decyl dimethyl benzyl ammonium hydroxide, didodecyl dimethyl ammonium hydroxide, dioctadecyl dimethyl ammonium hydroxide, tallow trimethyl ammonium hydroxide and coco trimethyl ammonium hydroxide as well as corresponding salts of these materials, fatty amines and fatty acid amides and their derivatives, basic pyridinium compounds, quaternary ammonium bases of benzimidazolines and polypropanolpolyethanol amines. Other representative examples of suitable cationic surfactants include alkylamine salts, sulphonium salts, and phosphonium salts.

Examples of suitable anionic surfactants include alkyl sulphates such as lauryl sulphate, polymers such as acrylates/alkyl (10 to 30 carbon atoms) acrylate crosspolymer alkylbenzenesulfonic acids and salts such as hexylbenzenesulfonic acid, octylbenzenesulfonic acid, decylbenzenesulfonic acid, dodecylbenzenesulfonic acid, cetylbenzenesulfonic acid and myristylbenzenesulfonic acid; the sulphate esters of monoalkyl polyoxyethylene ethers; alkylnapthylsulfonic acid; alkali metal sulforecinates, sulfonated glyceryl esters of fatty acids such as sulfonated monoglycerides of coconut oil acids, salts of sulfonated monovalent alcohol esters, amides of amino sulfonic acids, sulfonated products of fatty acid nitriles, sulfonated aromatic hydrocarbons, condensation products of naphthalene sulfonic acids with formaldehyde, sodium octahydroanthracene sulfonate, alkali metal alkyl sulphates, ester sulphates, and alkarylsulfonates. Anionic surfactants include alkali metal soaps of higher fatty acids, alkylaryl sulphonates such as sodium dodecyl benzene sulphonate, long chain fatty alcohol sulphates, olefin sulphates and olefin sulphonates, sulphated monoglycerides, sulphated esters, sulphonated ethoxylated alcohols, sulphosuccinates, alkane sulphonates, phosphate esters, alkyl isethionates, alkyl taurates, and alkyl sarcosinates. One example of an anionic surfactant is sold commercially under the name Bio-Soft N-300. It is a triethanolamine linear alkylate sulphonate composition marketed by the Stephan Company, Northfield, Ill.

The above surfactants may be used individually or in combination.

In one embodiment of the present invention, the polymerisation catalyst is selected such that the catalyst additionally functions as a surfactant for the emulsification step. Such a family of catalysts which can act as surfactants include, for example, acidic condensation catalysts, for example, DBSA.

Emulsification according to some embodiments of the present invention is carried out by combining the oil phase containing the branched polyorganosiloxane and the inert fluid with surfactant and water and mixing to form an emulsion. All or part of the water may be used in obtaining the emulsion. Intensity of agitation varies according to desired particle size. Typically, to achieve fine emulsion particle size, an initial small amount of water, for example, from 0.1% to 10% by weight per oil phase containing the branched polyorganosiloxane and the inert fluid, may be used to obtain the emulsion. Generally, the higher the intensity of shear, the lower the particle size achieved. After the desired particle size has been reached, the emulsion may be diluted with more water to achieve the desirable active content.

Alternatively, emulsification may be carried out by dispersing or metering the oil phase containing the branched polyorganosiloxane and the inert fluid into the aqueous phase containing the surfactants while under constant agitation to form an emulsion. The emulsion may subsequently be subjected to high shear to reduce particle size.

The emulsions produced by the methods of the present invention may have a wide variety of polysiloxane containing polymer concentrations, particle sizes and molecular weights, including novel materials having high concentrations of large particle polysiloxane containing polymer of high molecular weight. The particle size may be, for example, chosen within the range 0.1 to 1000 micrometres.

If desired, other materials may be added to either phase of the emulsions, for example, perfumes, fillers, relaxers, colorants, thickeners, preservatives, or active ingredients such as pharmaceuticals antifoams, freeze thaw stabilizers, inorganic salts to buffer pH, and thickeners

The emulsions of the present invention can generally have a branched organopolysiloxane loading in the range of about 1% to about 94% of the weight of the oil phase containing the branched organopolysiloxane and the inert fluid. Alternatively, the branched organopolysiloxane may be present in amounts from about 10% to about 90%, about 20% to about 80%, about 30% to about 70%, or about 40% to about 60% the weight of the oil phase. The branched organopolysiloxanes produced according to the methods of the present invention are particularly useful for personal care products. The branched organopolysiloxane product containing the inert fluid may be further dissolved in an organic solvent or emulsified in water if the branched organopolysiloxane formulation is required in solution or emulsion form.

The emulsions of the invention are useful in applications for silicone emulsions, for example, in personal care applications such as on hair, skin, mucous membrane or teeth. In these applications, the silicone is lubricious and will improve the properties of skin creams, skin care lotions, moisturisers, facial treatments such as acne or wrinkle removers, personal and facial cleansers such as shower gels, liquid soap, bar soaps hand sanitizers and wipes, bath oils, perfumes, fragrances, colognes, sachets, deodorants, sun protection creams, lotions, spray, stick and wipes, self tanning creams, lotions, spray and wipes, pre-shave and after shave lotions, after sun lotion and creams, anti-perspirant sticks, soft solid and roll-ons, hand sanitizers, shaving soaps and shaving lathers. It can likewise be use in hair shampoos, rinse-off and leave-on hair conditioners, hair styling aids, such as sprays, mousses and gels, hair colorants, hair relaxers, permanents, depilatories, and cuticle coats, for example to provide styling and conditioning benefits. In cosmetics, it function as a levelling and spreading agent for pigment in make-ups, colour cosmetics, compact gel, cream and liquid foundations (w/o and o/w emulsions, anhydrous), blushes, lipsticks, lip gloss, eye liners, eye shadows, mascaras, make up removers, colour cosmetic removers and powders. It is likewise useful as a delivery system for oil and water soluble substances such as vitamins, fragrances, emollients, colorants, organic sunscreens, ceramides, pharmaceuticals and the like. When compounded into sticks, anhydrous and aqueous gels, o/w and w/o creams and lotions, aerosols and roll-ons, the emulsions of this invention impart a dry silky-smooth payout.

In step (iv) of the methods of the present invention, the optional applying shear. The emulsions of the present invention can be further sheared to reduce drop size by using any conventional mixers or high shear devices such as those operated by impellers, rotor stators, high pressure valves and cavitation processors. Commercial examples include the Lightnin® mixers, Ross mixers (by Charles Ross & Son Company), Ultra-Turrax® dispersers, colloid mills, Microfluidizer® processors, and Sonolator™ homogenizers.

For use in personal care products, the branched organopolysiloxane product may be, for example, dissolved in an organic solvent or emulsified in water using an anionic, cationic, amphoteric and/or non-ionic surfactant. If a personal care product, for example a cosmetic such as a skin cream, is required in organic solution form it may be convenient to react the branching agent and substantially linear organopolysiloxane in solution in the organic solvent to be used in the personal care product.

Personal care formulations containing the branched polyorganosiloxane may contain various additives known in such formulations, for example perfumes, sunscreens, antioxidants, vitamins, drugs, biocides, pest repellents, catalysts, natural extracts, peptides, warming effect and cooling agents, fillers, colouring agents such as dyes, pigments and shimmers, heat stabilizers, flame retardants, UV stabilizers, fungicides, biocides, thickeners, preservatives, antifoams, freeze thaw stabilizers, or inorganic salts to buffer pH.

When a personal care product containing a branched organopolysiloxane according to the invention is applied to the skin or hair, the product is generally more resistant to washing off than a similar product containing a linear organopolysiloxane of similar molecular weight.

When used in personal care products, the emulsions are generally incorporated in amounts of about 0.01% to about % by weight, or 0.1% to 25% by weight of the personal care product. The emulsions are added to conventional ingredients for the personal care product chosen. Thus, the emulsions can be mixed with deposition polymers, surfactants, detergents, antibacterials, anti-dandruffs, foam boosters, proteins, moisturising agents, suspending agents, pacifiers, perfumes, colouring agents, plant extracts, polymers, and other conventional care ingredients.

Beyond personal care, the emulsion of the present invention are useful for numerous other applications such as paints, construction applications, textile fibre treatment, leather lubrication, fabric softening, fabric care in laundry applications, healthcare, homecare, release agents, water based coatings, oil drag reduction, particularly in crude oil pipelines, lubrication, facilitation of cutting cellulose materials, and in many other areas where silicones are conventionally used. The silicone organic copolymers have particular advantages in oil drag reduction resulting from increased compatibility with hydrocarbon fluids.

Having described the invention with reference to certain embodiments, other embodiments will become apparent to one skilled in the art from consideration of the specification. The invention is further defined by reference to the following examples describing the preparation of the emulsions and methods of the invention. It will be apparent to those skilled in the art that many modifications, both to materials and methods, may be practiced without departing from the scope of the invention

The invention is illustrated by the following Examples, in which parts and percentages are by weight. The molecular weight of the siloxanes in the mixtures was determined by gel permeation chromatography (GPC). The analyses have been performed by GPC (Alliance Waters 2690) using triple detection (Refractive index detector, Viscometer and Light Scattering Detectors) and toluene as solvent. Molecular weight averages were determined by universal calibration relative to a triple detection calibration realized on a single point using polystyrene narrow standard (Mw 70,950 g/mol).

### EXAMPLES

### Example 1

500 parts dimethylhydroxyl-terminated polydimethylsiloxane having a viscosity of 70 mPa·s at 25° C., a Mn of 2500 g/mol and a Mw of 3500 g/mol was mixed with 500 parts Hydroseal G 250H hydrocarbon oil (sold by Total), and 2 parts methyltrimethoxysilane (MTM). 15 parts per million (ppm) of an ionic phosphazene [Cl(PCl₂═N)ₓPCl₃]+[PCl₆]⁻ (x is 1 to 11) diluted in dichloromethane was added as catalyst. The polymerisation was carried out in a 1 liter glass reactor (IKA) at 70° C. under vacuum. The polymerisation was stopped after 5 minutes by the addition of 0.04 parts trihexylamine. A branched polydimethylsiloxane polymer, mixed with the hydrocarbon oil, was produced.

### Example 2

Another branched polydimethylsiloxane was produced by replacing the Hydroseal G 250H of Example 1 with Lytol™, a white mineral oil, supplied by Sonneborn and reaction time was 24 min.

Example

| | 1 | 2 |
|---|---|---|
| Mn (kg/mol) | 155 | 87 |
| Mw (kg/mol) | 1007 | 570 |
| PI | 6.5 | 6.6 |
| Viscosity (mPa · s) | 67800 | 63100 |

### Example 3

The branched polymers of Examples 1 and 2 were used to prepare emulsions. 200 g of the polymer blend from Example 1 or 2 was mixed with C12-13 Pareth-4 and C12-13 Pareth-23 in a SpeedMixer™ DAC 600 FVZ for 30 seconds at 2700 rpm. 5 wt % of the total water was added and content mixed for 2 minutes at 2700 rpm. The rest of the water was added incrementally and content mixed for 30 seconds at 2700 rpm upon each addition. Biocide was added and content mixed for 30 seconds at 2700 rpm. The emulsion formulations and properties are listed in table below. Emulsion particle size was measured using a Malvern Mastersizer™ 2000; volume averaged values are reported.

| | | | | |
|---|---|---|---|---|
| Product from Ex 1 | 50.5 | wt % | | |
| Product from Ex 2 | | | 50.4 | wt % |
| C12-13 Pareth-4 | 1.82 | wt % | 1.82 | wt % |
| C12-13 Pareth-23 | 2.05 | wt % | 2.05 | wt % |
| Water | 45.5 | wt % | 45.5 | wt % |
| Biocide* | 0.08 | wt % | 0.07 | wt % |
| Dv0.5 | 1.628 | micron | 1.486 | micron |
| Dv0.9 | 4.066 | micron | 3.037 | micron |

*Biocide: mixture of 5-chloro-2-methyl-4-isothiazolin-3-one at 1.13% active by weight and 2-methyl-4-isothiazolin-3-one at 0.37% active by weight.

### Example 4

In this Example, 300 parts dimethylhydroxyl-terminated polydimethylsiloxane was mixed with 300 parts isohexadecane, and 1 part methyltrimethoxysilane (MTM). 5 parts per million (ppm), with respect to dimethylhydroxyl-terminated polydimethylsiloxane, of neutral, partially hydrolyzed phosphazene, Cl(PCl₂═N)ₙ—P(O)Cl₂ or HO(PCl₂═N)ₙ—P(O)Cl₂ (n is 1 to 10) diluted in propylene carbonate was added as catalyst. The polymerisation was carried out in a 1 liter glass reactor (ESCO) at 70° C. under vacuum. The polymerisation was stopped after 20 minutes by the addition of 25 ppm (with respect to dimethylhydroxyl-terminated polydimethylsiloxane) trihexylamine diluted in isohexadecane. A branched polydimethylsiloxane polymer, mixed with the hydrocarbon oil, was produced. The reaction product was used to make an emulsion.

The emulsion was made as follows. To a 60 g mixing cup of a SpeedMixer™ DAC 150 FVZ 25 grams of the reaction product, 0.37 grams Lutensol™ XP79, 0.60 grams Arquad™ 16-29 and 0.61 grams de-ionized water. The content was mixed at 3500 rpm for 30 seconds to form a white emulsion. The emulsion was further mixed at 3500 rpm for one minute at a time and a total of four times to reduce particle size. To the emulsion was added 1 gram de-ionized water followed by mixing for 30 seconds. Another 22.3 grams of de-ionized water was added followed by mixing. This arrived at an emulsion having a volume averaged median particle size of 4.1 microns.

### Example 5

In this Example, 300 parts dimethylhydroxyl-terminated polydimethylsiloxane was mixed with 300 parts Lytol™, a white mineral oil, supplied by Sonneborn, and 1 part methyltrimethoxysilane (MTM). 5 parts per million (ppm), with respect to dimethylhydroxyl-terminated polydimethylsiloxane, of ionic phosphazene [Cl(PCl₂═N)ₓPCl₃]⁺[PCl₆]⁻ (x is 1 to 10) diluted in dichloromethane was added as catalyst. The polymerisation was carried out in a 1 liter glass reactor (ESCO) at 70° C. under vacuum. The polymerisation was stopped after 20 minutes by the addition of 25 ppm (with respect to dimethylhydroxyl-terminated polydimethylsiloxane) trihexylamine diluted in isohexadecane. A branched polydimethylsiloxane polymer, mixed with the hydrocarbon oil, was produced. The reaction product was used to make an emulsion.

The emulsion was prepared as follows. In the vessel of a Ross PowerMix™ model PD-1/2 was loaded 496 grams of the reaction product, 6.0 grams Renex™ 36, 12.04 grams Arquad 16-29 and 12.90 grams de-ionized water. The content was mixed at a disperser speed of 342 rpm and a planetary speed of 40 rpm for 1 minute to form a coarse emulsion. The emulsion was sheared for an additional 5 minutes at a disperser speed of 1026 rpm and a planetary speed of 40 rpm. This arrived at an emulsion having a volume averaged median particle size of 3.17 microns.

### Example 6

In this Example, 300 parts dimethylhydroxyl-terminated polydimethylsiloxane was mixed with 300 parts isohexadecane, and 1.07 parts methyltrimethoxysilane (MTM). 5 parts per million (ppm), with respect to dimethylhydroxyl-terminated polydimethylsiloxane, of ionic phosphazene [Cl(PCl₂═N)ₓPCl₃]+[PCl₆]⁻ (x is 1 to 10) diluted in dichloromethane was added as catalyst. The polymerisation was carried out in a 1 liter glass reactor (ESCO) at 70° C. under vacuum. The polymerisation was stopped after 11 minutes by the addition of 25 ppm (with respect to dimethylhydroxyl-terminated polydimethylsiloxane) trihexylamine diluted in isohexadecane. A branched polydimethylsiloxane polymer, mixed with the hydrocarbon oil, was produced. The reaction product was used to make an emulsion.

The emulsion was made as follows. In a 1 liter stainless steel beaker was loaded 180 grams of the reaction product from Example 5, 6.0 grams Brij® L4, 11.29 grams Brij™ L23 (69% active in water) and 20.42 grams de-ionized water. The content was mixed using a Premier Mill Laboratory Dispersator equipped with a Cowles blade at a speed of 300 rpm for 1 minute to form a coarse emulsion. The emulsion was sheared for an additional 1 hour at 1200 rpm. The emulsion was diluted with 82.32 grams de-ionized water with slow agitation. Finally 0.45 grams of phenoxyethanol was added and mixed into the emulsion. This arrived at an emulsion having a volume averaged median particle size of 0.94 microns.

### Example 7

To a 60 g mixing cup of a SpeedMixer™ DAC 150 FVZ was loaded 18 grams of an α,ω-hydroxyl terminated polydimethylsiloxane of viscosity 70 centipoise, 2 grams of sunflower oil, and 0.07 grams tetraethyl orthosilicate. The content was mixed at 3500 rpm for 30 seconds. To the content was added 0.6 grams dodecylbenzenesulfonic acid and the content was mixed at 3500 rpm for 30 seconds. The mixture was let stand for 10 minutes during which it became noticeably thicker. 0.27 grams of triethanolamine was then added to the mixture and the content mixed at 3500 rpm for 30 seconds. Steady-shear viscosity was measured using a Brookfield DV-III model using cone-and-plane with a CPE-52 spindal to be 43,600 centipoise at a shear rate of 4 sec⁻¹.

To the above mixture containing silicone and sunflower oil was added 0.52 grams Brij® L4 and 2 grams of de-ionized water followed by mixing for 2 minute at 3500 rpm. This produced a white thick emulsion. The emulsion was then diluted with an additional 11 grams of de-ionized water. The final emulsion measured a volume averaged median particle size of 1.39 microns using a Malvern Mastersizer™.

1. A method of making an oil-in-water emulsion comprising a branched organopolysiloxane, the method comprising:
(i) preparing a branched organopolysiloxane comprising reacting a branching agent with a substantially linear organopolysiloxane containing at least one hydroxyl or hydrolyzable group bonded to silicon in the presence of an inert fluid, a catalyst and optionally an end-blocking agent to obtain a solution or dispersion containing the branched organopolysiloxane, and a portion of the inert fluid;
(ii) quenching the reaction, if required;
(iii) adding water and one or more surfactants to the solution or dispersion containing the branched organopolysiloxane and mixing to form the oil-in-water emulsion; and
(iv) optionally applying shear to the emulsion to reduce particle size.

2. The method according to claim 1, wherein the substantially linear organopolysiloxane is of general formula
X¹-A-X² (1)
wherein X¹ and X² are independently selected from silicon containing groups which contain hydroxyl or hydrolyzable substituents, and A represents a polymer chain of formula
—(R²₂SiO)— (2)
wherein each R² is independently an organic group such as a hydrocarbon group having from 1 to 18 carbon atoms, a substituted hydrocarbon group having from 1 to 18 carbon atoms or a hydrocarbonoxy group having 1 to 18 carbon atoms.

3. The method according to claim 1, wherein the Mw of the branched organopolysiloxane is from about 10,000 to about 10,000,000 g/mol.

4. The method according to claim 1, wherein the ratio of the linear organopolysiloxane to the inert fluid is from about 1:10 to about 10:1 by weight.

5. The method according to claim 1, wherein the ratio of the linear organopolysiloxane to the branching agent is from about 10:1 to about 1000:1 by weight.

6. The method according to claim 1, further comprising diluting the oil-in-water emulsion by adding more water.

7. The method according to claim 1, wherein the substantially linear organopolysiloxane comprises terminal hydroxyl groups bonded to silicon.

8. The method according to claim 1, wherein the branching agent is of general formula
R¹Si(OR)₃
wherein R is selected from the group consisting of hydrogen, an alkyl group of 1 to 6 carbon atoms, an alkenyl group of 2 to 6 carbon atoms, a saturated or unsaturated cyclic group of 3 to 6 carbon atoms, an acyl group of 1 to 6 carbon atoms, and an aryl-carbonyl group wherein the aryl is of 6 to 10 carbon atoms, wherein the alkyl, alkenyl, cyclic or aryl group is unsubstituted or substituted with one or more groups selected from an alkyl group of 1 to 6 carbon atoms, a hydroxy, an alkoxy group of 1 to 6 carbon atoms, a cycloalkyl group of 3 to 6 carbon atoms, halogen and cyano, and R¹ is a monovalent substituted or unsubstituted hydrocarbon group of 1 to 18 carbon atoms or an alkoxy group of 1 to 6 carbon atoms.

9. The method according to claim 8, wherein R is CH₃C(O)—, CH₃CH₂C(O)—, HOCH₂CH₂—, CH₃OCH₂CH₂—, or C₂H₅OCH₂CH₂—.

10. The method according to claim 1, wherein the branching agent comprises a tetraalkoxysilane.

11. The method according to claim 1, wherein the branching agent comprises a partially condensed alkoxysilane containing on average more than two alkoxy groups per molecule bonded to silicon.

12. The method according to claim 1, wherein the catalyst is a phosphazene catalyst.

13. The method according to claim 12, wherein the phosphazene catalyst is a perchlorooligophosphazenium salt of the formula
[Cl₃P—(N═PCl₂)ₙCl]⁺Z⁻
wherein n has an average value in the range 1 to 10 and Z represents an anion of the formula MXᵥ₊₁ in which M is an element having an electronegativity on Pauling's scale of from 1.0 to 2.0 and valency v and X is a halogen atom.

14. The method according to claim 12, wherein the phosphazene catalyst is an oxygen-containing chlorophosphazene of the formula
Cl(PCl₂═N)ₙ—P(O)Cl
or
HO(PCl₂═N)ₙ—P(O)Cl₂
wherein n has an average value in the range 1 to 10.

15. The method according to claim 12, wherein the phosphazene catalyst is an oxygen-containing chlorophosphazene containing organosilicon radicals of the formula
R⁵₃SiO(PCl₂═N)ₙ—P(O)Cl₂
wherein each R⁵ represents a monovalent substituted or unsubstituted hydrocarbon group having 1 to 18 carbon atoms and n has an average value in the range 1 to 10.

16. The method according to claim 12, wherein the phosphazene catalyst is hydrolyzed phosphazene catalyst or a non-hydrolyzed phosphazene.

17. The method according to claim 1, wherein the inert fluid is a liquid linear or branched paraffin containing 12 to 40 carbon atoms.

18. The method according to claim 1, wherein the inert fluid is a natural oil.

19. An emulsion prepared according to claim 1.

20. Use of the oil-in-water emulsions comprising a branched organopolysiloxane according to claim 19 or prepared according to claim 1 in a personal care product applied to skin or hair.
